(19) United States
(12) Patent Application Publication
(10) Pub. No.: US 2026/0265194 A1
(43) Pub. Date: Sep. 10, 2026

(54) COMPOUNDS FOR THE TREATMENT OF HIV

(71) Applicants: THE TRUSTEES OF THE UNIVERSITY OF PENNSYLVANIA, Philadelphia, PA (US); DANA-FARBER CANCER INSTITUTE, Boston, MA (US)

(72) Inventors: Amos B. SMITH, III, Merion Station, PA (US); Christopher James FRITSCHI, Philadelphia, PA (US); Joseph G. SODROSKI, Medford, MA (US); Navid MADANI, Newton, MA (US); Saumya ANANG, Allston, MA (US); Derek YANG, Sunnyvale, CA (US); Hung-Ching CHEN, Philadelphia, PA (US)

(21) Appl. No.: 18/867,877

(22) PCT Filed: May 31, 2023
(86) PCT No.: PCT/US2023/067700

§ 371 Date: Nov. 21, 2024

Related U.S. Application Data
(60) Provisional application No. 63/347,477, filed on May 31, 2022.

(51) Int. Cl. C07D 209/40; A61K 31/4045; A61P 31/18

(52) U.S. Cl. CPC C07D 209/40; A61K 31/4045; A61P 31/18

(57) ABSTRACT

Compositions and methods for treating HIV using 2-(guanidinomethyl)-6-(aminomethyl)-indolinyl derivatives that sensitize HIV-I infected cells to antibody-dependent cellular cytotoxicity, comprising administering to the mammal in need thereof a compound. The compounds of the disclosure provide viral entry inhibition of JR-FL and other resistant HIV-I strains, host antibody recognition and CoRBS exposure of Env, and kill infected cells through ADCC.

### CROSS REFERENCE OF RELATED APPLICATIONS

The present application claims priority to and the benefit of U.S. patent application No. 63/347,477, filed May 31, 2022 entitled “Compounds For The Treatment Of HIV”. All foregoing applications are incorporated herein by reference in their entireties for any and all purposes.

### GOVERNMENT RIGHTS

This invention was made with government support under 5P01AI150471-24 awarded by the National Institutes of Health. The government has certain rights in the invention.

### TECHNICAL FIELD

The disclosure is directed to compounds that are useful in treating HIV.

### BACKGROUND

The HIV pandemic continues to be a global health issue, with 1.5 million new infections reported by the World Health Organization (WHO) in 2020. Encouragingly, of the 38 million individuals infected worldwide, 62% of those living with HIV are receiving some form of treatment. Although there are treatment options available (ca. ART), the control of viral rebound following cessation of antiretroviral treatment and the prevention of HIV-1 transmission remain elusive goals.

In order to recognize cells for infection, the HIV virus expresses a trimeric glycoprotein (Env) that decorates the virion surface. When Env comes into contact with a CD4 receptor on the surface of a T-cell, the Env transitions to an “open” conformation. Following this initial engagement, further conformational changes occur within the Env protein which ultimately exposes a fusion peptide. This peptide then inserts into the host cell membrane and results in viral/host cell membrane fusion. Once fused, the virus releases its genetic information and begins replication. Importantly, Env adopts a “closed” conformation when CD4 is not present. This prevents the host's immune system from recognizing the virus as a foreign body. Compounds that mimic the interaction between Env and CD4, to activate the trimer prematurely and stabilize the “open” conformation, lead to an inactive state of Env, thus inhibiting the viral entry process.

Compounds that mimic the interaction between Env and CD4 are needed.

### SUMMARY

The disclosure is directed to compounds of Formula I as well as stereoisomers thereof and pharmaceutically acceptable salts thereof, wherein R is C₀₋₆alk-aryl, substituted C₀₋₆alk-aryl, C₁₋₆alkyl, substituted C₁₋₆alkyl, C₁₋₆haloalkyl, C₂₋₆alkenyl, or substituted C₂₋₆alkenyl; R₁ is F, Cl, or Br; R₂ is F, Cl, or Br; R₃ is H or C₁₋₆alkyl; and R₄ is H or C₁₋₆alkyl. Methods of making and using these compounds are also described.

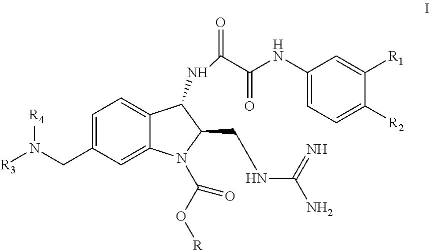

### BRIEF DESCRIPTION OF THE DRAWINGS

The foregoing summary, as well as the following detailed description, will be better understood when read in conjunction with the appended drawings, which show exemplary embodiments for the purposes of illustration.

FIG. 1 depicts that increasing the potency of CD4-mimetic compounds results in increased breadth against diverse non-Clade AE HIV-1 strains (n=19).

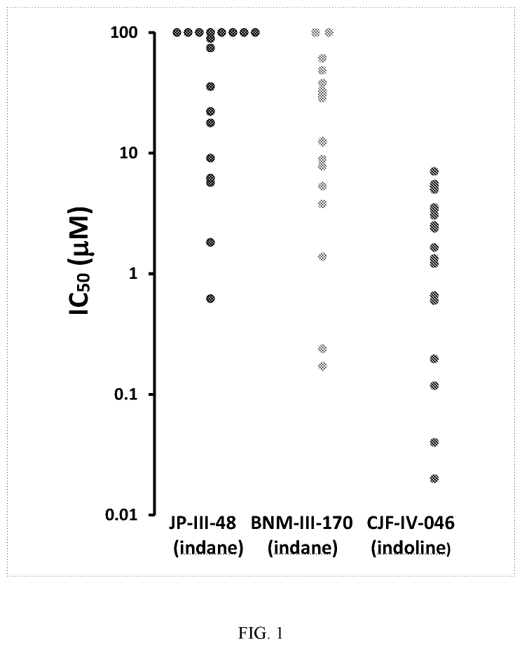

FIG. 2 depicts normalized JR-FL IC₅₀ of compounds of the disclosure compared to BNM-III-170.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The term “administering” means either directly administering to the subject a compound, or composition comprising that compound, of the present invention. In other aspects, “administering” refers to administering a prodrug, derivative or analog or a compound of the present invention, which will form an equivalent amount of the compound within the body.

As used herein, the term “stereoisomers” refers to compounds which have identical chemical constitution but differ with regard to the arrangement of the atoms or groups in space, e.g., enantiomers, diastereomers, tautomers.

The terms “patient” and “subject” is used interchangeably throughout the specification to describe an animal, for example, a mammal, to whom treatment, including prophylactic treatment, with the compositions according to the present disclosure is provided. Mammals that can be treated using the methods of the disclosure include rodents such as mice, rats, rabbits, guinea pigs, and the like, as well as domesticated animals such as dogs, cats, and farm animals such as a horse, cow, sheep, etc. In other aspects, the mammal is a human.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

As employed above and throughout the disclosure the term “therapeutically effective amount” refers to an amount effective, at dosages, and for periods of time necessary, to achieve the desired result with respect to the treatment of the relevant disorder, condition, or side effect. It will be appreciated that the effective amount of compounds of the present invention will vary from subject to subject.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. See for example, Remington, J. P. (2020). Remington, the science and practice of pharmacy, Elsevier Science.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “-C₀alk-” refers to a bond.

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, the alkyl group is a C₁-C₄ alkyl.

The terms “halo” or “halogen”, by itself or as part of another substituent, means a fluorine, chlorine, bromine, or iodine atom.

The term “haloalkyl” refers to an alkyl moiety wherein one or more hydrogens has been replaced with one or more halogen atoms. Examples include CF₃, CH₂CF₃, and the like.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1, 2, 3,4-tetrahydronaphthyl, and the like.

As used herein, the term “alkenyl”, by itself or as part of another substituent, means, unless otherwise stated, carbon chains which contain at least one carbon-carbon double bond, and which may be linear, branched, cyclic, or combinations thereof. Examples of alkenyl groups include vinyl, allyl, isopropenyl, pentenyl, hexenyl, heptenyl, 1-propenyl, 2-butenyl, 2-methyl-2-butenyl, and the like. Alkenyl groups may be optionally substituted as provided herein. In some embodiments, the alkenyl group is a C₂₋₆alkenyl. In some embodiments, the alkenyl group is a C₂₋₄alkenyl.

The term “substituted”, as used in combination with a substituent defined herein, means that the substituent is substituted with one or more suitable functional groups or other substituents as provided herein. For example, a substituent may be optionally substituted with one or more of: halo, cyano, C₁₋₆ alkyl, aryl, benzyl, or the like.

The disclosure is directed to compounds of Formula I, stereoisomers of Formula I, and pharmaceutically acceptable salts thereof. While not wishing to be bound by any particular theory, the compounds of Formula I are believed to be CD4 mimetic compounds (CD4mc) that open Env, thus allowing enhanced recognition and binding by antibodies resulting in the sensitization of HIV-1 infected cells to ADCC.

The disclosure is directed to compounds of Formula I, as well as stereoisomers thereof, and pharmaceutically acceptable salts thereof:

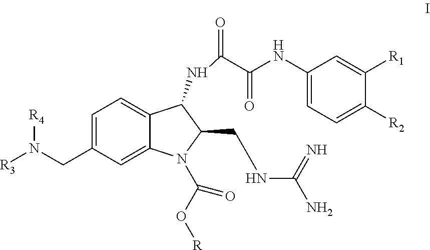

According to the disclosure, R is C₀₋₆alk-aryl, substituted C₀₋₆alk-aryl, C₁₋₆alkyl, substituted C₁₋₆alkyl, C₁₋₆haloalkyl, C₂₋₆alkenyl, or substituted C₂₋₆alkenyl; R₁ is F, C₁, or Br; R₂ is F, Cl, or Br; R₃ is H or C₁₋₆alkyl; and R₄ is H or C₁₋₆alkyl.

According to the disclosure, R is C₀₋₆alk-aryl, substituted C₀₋₆alk-aryl, C₁₋₆alkyl, substituted C₁₋₆alkyl, C₁₋₆haloalkyl, C₂₋₆alkenyl, or substituted C₂₋₆alkenyl.

In some aspects, R is C₀₋₆alk-aryl. In some aspects, R is C₀₋₃alk-aryl. In some aspects R is C₀alk-aryl. In some aspects, R is C₁alk-aryl. In some aspects R is C₂alk-aryl. In some aspects R₂ is C₀₋₆alk-phenyl. In some aspects R is —CH₂-phenyl.

In some aspects, R is substituted C₀₋₆alk-aryl. In these aspects, the aryl moiety may be substituted. In other aspects, when present, the C₁₋₆alk moiety is substituted.

In other aspects, the aryl and the C₁₋₆alk moiety are each independently substituted. In some aspects R is substituted C₀₋₃alk-aryl. In some aspects R is substituted C₀alk-aryl. In some aspects R is substituted C₁alk-aryl. In some aspects R is substituted C₂alk-aryl. In some aspects R is substituted C₀₋₆alk-phenyl. In some aspects R is —CH₂-phenyl. In some aspects R is C₁alkaryl substituted with one or more of F, Br, NO₂. In some aspects R is substituted C₁alkaryl, preferably pentafluorobenzyl, 4-bromobenzyl, 4-nitrobenzyl, or 3-methyl-4-nitrobenzyl. In some aspects R is C₀alk-aryl, preferably phenyl.

In some aspects, R is C₁₋₆alkyl. In some aspects, R is C₁₋₃alkyl. In some aspects R is C₁alkyl. In some aspects R is C₂alkyl. In some aspects R is C₃alkyl. In some aspects R is C₄alkyl. In some aspects R is C₅alkyl. In some aspects R is C₆alkyl. In some aspects R is —CH₃. In some aspects R is ethyl, n-propyl, or 2,3-dihydroxpropyl such as (S)-2,3-dihydroxypropyl.

In some aspects, R is substituted C₁₋₆alkyl. In some aspects, R is substituted C₁₋₃alkyl. In some aspects R is substituted C₁alkyl. In some aspects R is substituted C₂alkyl.

In some aspects R is substituted C₃alkyl. In some aspects R is substituted C₄alkyl. In some aspects R is substituted C₅alkyl. In some aspects R is substituted C₆alkyl. In some aspects R is —CH₃.

In some aspects R, is C₁₋₆haloalkyl. In some aspects, R is C₁₋₃haloalkyl. In some aspects R is C₁haloalkyl. In some aspects R is C₂haloalkyl. In some aspects R is C₃haloalkyl. In some aspects R is —CF₃. In some aspects R is —CH₂CF₃.

In some aspects R is C₂₋₆alkenyl. In some embodiments, the alkenyl group is a C₂₋₄alkenyl. In some aspects R is C₂alkenyl. In some aspects R is C₃alkenyl. In some aspects R is C₄alkenyl. In some aspects R is C₅alkenyl. In some aspects R is C₆alkenyl. In some aspects R is allyl.

In some aspects R is substituted C₂₋₆alkenyl. In some aspects R is substituted C₂alkenyl. In some aspects R is substituted C₃alkenyl. In some aspects R is substituted C₄alkenyl. In some aspects R is substituted C₅alkenyl. In some aspects R is substituted C₆alkenyl. In some aspects R is substituted allyl.

According to the disclosure, R₁ is halo, for example F, Cl, or Br. In some aspects R₁ is F. In some aspects R₁ is C₁. In some aspects R₁ is Br.

According to the disclosure, R₂ is halo, for example F, Cl, or Br. In some aspects R₂ is F. In some aspects R₂ is C₁. In some aspects R₂ is Br.

According to the disclosure R₃ is H, C₁₋₆alkyl. In some aspects R₃ is H. In other aspects R₃ is C₁₋₆alkyl. In some aspects R₃ is C₁alkyl. In some aspects R₃ is C₂alkyl. In some aspects R₃ is C₃alkyl. In some aspects R₃ is C₄alkyl. In some aspects R₃ is C₅alkyl. In some aspects R₃ is C₆alkyl. In some aspects R₃ is —CH₃.

According to the disclosure R₄ is H or C₁₋₆alkyl. In some aspects R₄ is H. In other aspects R₄ is C₁₋₆alkyl. In some aspects R₄ is C₁alkyl. In some aspects R₄ is C₂alkyl. In some aspects R₄ is C₃alkyl. In some aspects R₄ is C₄alkyl. In some aspects R₄ is C₅alkyl. In some aspects R₄ is C₆alkyl. In some aspects R₃ is —CH₃.

In some embodiments, the compound of Formula I is CJF-IV-046, a stereoisomer of compound CJF-IV-046, or a pharmaceutically acceptable salt thereof

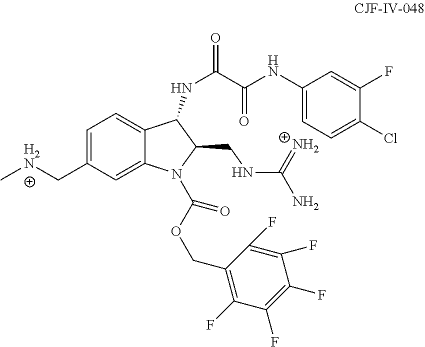

In some embodiments, the compound of Formula I is CJF-III-192, a stereoisomer of compound CJF-III-192, or a pharmaceutically acceptable salt thereof.

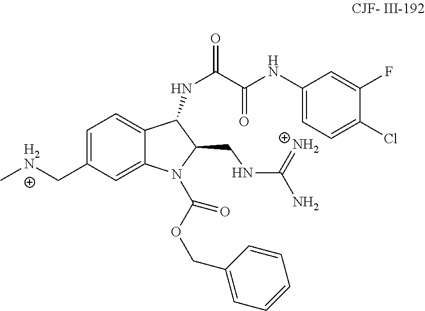

In some embodiments, the compound of Formula I is CJF-III-288-B, a stereoisomer of compound CJF-III-288-B, or a pharmaceutically acceptable salt thereof.

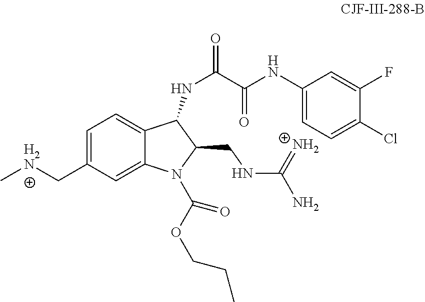

In some embodiments, the compound of Formula I is CJF-IV-046-C, a stereoisomer of compound CJF-IV-046-C, or a pharmaceutically acceptable salt thereof.

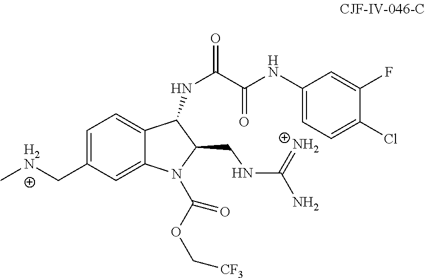

In some embodiments, the compound of Formula I is CJF-III-288-C, a stereoisomer of compound CJF-III-288-C, or a pharmaceutically acceptable salt thereof.

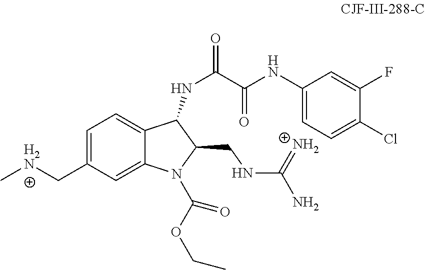

In some embodiments, the compound of Formula I is CJF-III-280, a stereoisomer of compound CJF-III-280, or a pharmaceutically acceptable salt thereof.

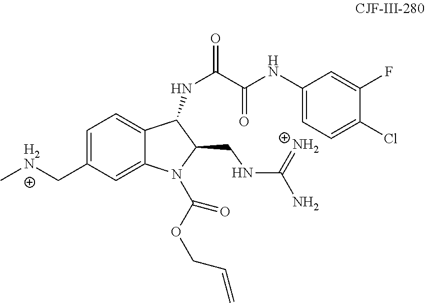

In some embodiments, the compound of Formula I is selected from the following table.

TABLE 1

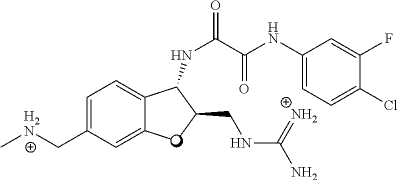

| Compound | 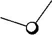 |
|---|---|
| CJF-III-192 | 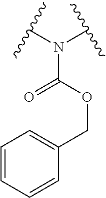 [SMILES: *N(*)C(=O)OCc1ccccc1] |
| CJF-IV-046 | 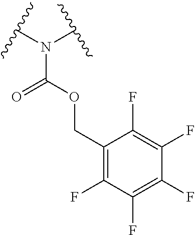 [SMILES: *N(*)C(=O)OCc1c(F)c(F)c(F)c(F)c1F] |
| CJF-III-288-D | 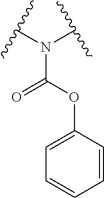 [SMILES: *N(*)C(=O)Oc1ccccc1] |
| CJF-III-214 | 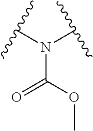 [SMILES: *N(*)C(=O)OC] |
| CJF-III-288-C | 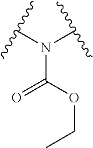 [SMILES: *N(*)C(=O)OCC] |
| CJF-IV-046-C | 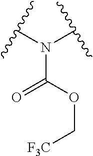 [SMILES: *N(*)C(=O)OCC(F)(F)F] |
| CJF-III-288-B | 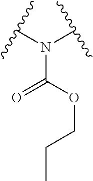 [SMILES: *N(*)C(=O)OCCC] |
| CJF-III-280 | 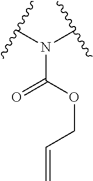 [SMILES: *N(*)C(=O)OCC=C] |
| CJF-III-288-A | 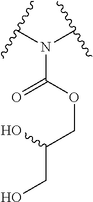 [SMILES: *N(*)C(=O)OCC(O)CO] |
| CJF-IV-050-A | 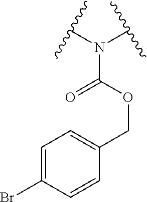 [SMILES: *N(*)C(=O)OCc1ccc(Br)cc1] |
| CJF-IV-050-C | 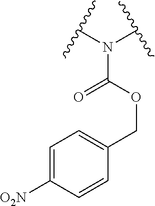 [SMILES: *N(*)C(=O)OCc1ccc([N+](=O)[O-])cc1] |
| CJF-IV-050-D | 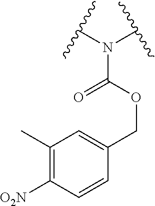 [SMILES: *N(*)C(=O)OCc1ccc([N+](=O)[O-])c(C)c1] |

In some aspects, the compounds of the disclosure are useful in treating or preventing HIV-1 replication in a mammal. In these aspects, a compound of the disclosure is administered to the mammal in need of HIV-1 replication treatment or prevention.

In some aspects, the compounds of the disclosure are useful in preventing transmission of HIV-1 to a mammal. In these aspects, a compound of the disclosure is administered to the mammal in need.

In some aspects, the compounds of the disclosure are used in combination with one or more antiretrovirals that are known in the art.

Compounds of the disclosure can be combined with pharmaceutically acceptable excipients to produce pharmaceutical compositions. Pharmaceutical compositions are typically formulated to provide a therapeutically effective amount of a compound of the present disclosure as the active ingredient, or a stereoisomer thereof, or a pharmaceutically acceptable salt, thereof in combination with one or more pharmaceutically acceptable excipients, carriers, including inert solid diluents and fillers, diluents, including sterile aqueous solution and various organic solvents, permeation enhancers, solubilizers and adjuvants.

Compounds of the disclosure can be prepared according to the following scheme, in combination with knowledge of those skilled in the art of chemical synthesis.

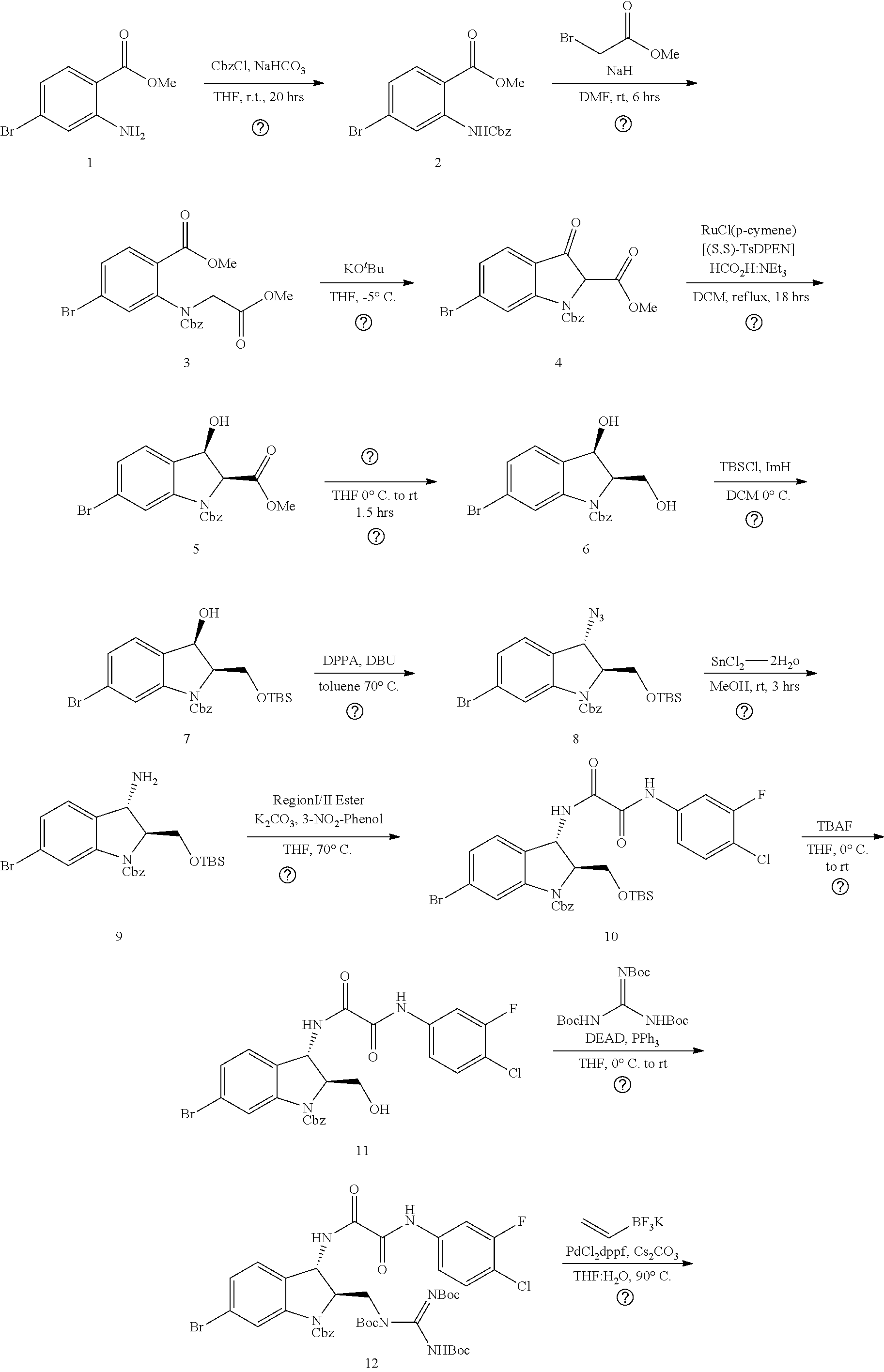

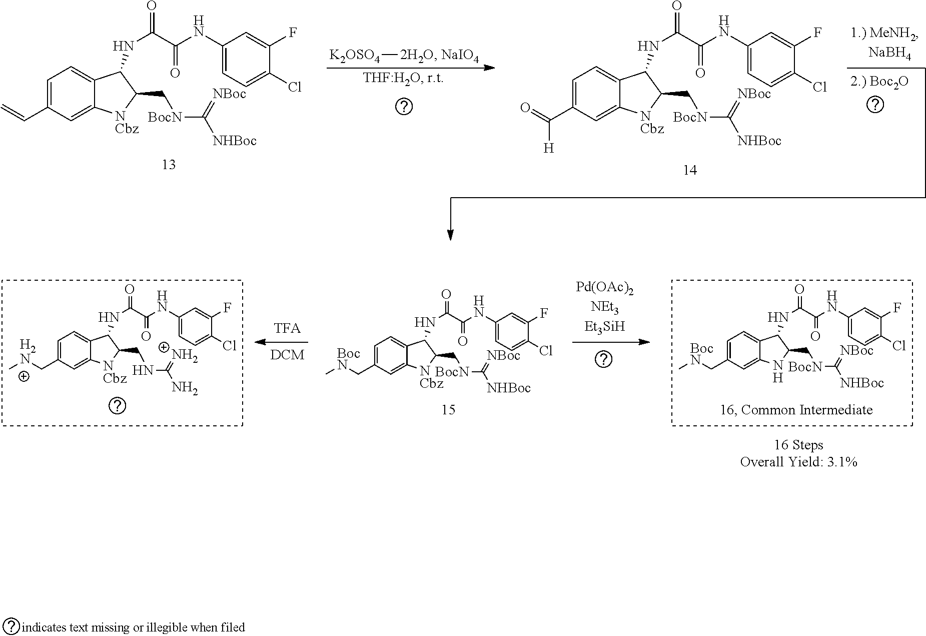

From the common intermediate 16, compounds of the disclosure can be prepared via a 3-step acylation, substitution, and deprotection sequence according to according to the following scheme, in combination with knowledge of those skilled in the art of chemical synthesis.

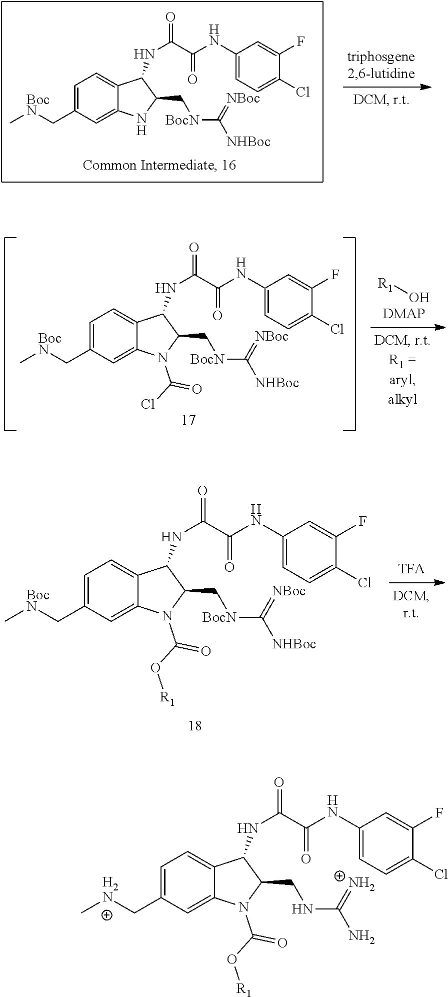

### General Information

All solvents were reagent or high-performance liquid chromatography (HPLC) grade. Anhydrous CH₂C₁₂ and THF were obtained from the Pure Solve™ PS-400 system under an argon atmosphere. All reagents were purchased from commercially available sources and used as received. Reactions were magnetically stirred under a nitrogen or argon atmosphere, unless otherwise noted and reactions were monitored by Thin layer chromatography (TLC) was performed on pre-coated silica gel 60 F-254 plates (40-55 micron, 230-400 mesh) and visualized by UV light. Yields refer to chromatographically and spectroscopically pure compounds. Optical rotations were measured on a JASCO P-2000 polarimeter. Proton (H) and carbon (¹³C) NMR spectra were recorded on a Bruker Avance III 500-MHz spectrometer or a Bruker NEO600 600-MHz spectrometer. Chemical shifts (δ) are reported in parts per million (ppm) relative to chloroform (δ 7.26), methanol (δ 3.31), or acetone (δ 2.05) for ¹H NMR, and chloroform (δ 77.2) methanol (δ 49.15), or acetone (δ29.92) for ¹³C NMR. High resolution mass spectra (HRMS) were recorded at the University of Pennsylvania Mass Spectroscopy Service Center on either a VG Micromass 70/70H or VG ZAB-E spectrometer. Analytical HPLC was performed with a Waters HPLC-MS system, consisting of a 515 pump and Sunfire C₁₈ reverse phase column (20 μL injection volume, 5 μm packing material, 4.5×50 mm column dimensions) with detection accomplished by a Micromass ZQ mass spectrometer and 2996 PDA detector. SFC analyses were performed with a JASCO system equipped with a PU-280-CO₂ plus CO₂ Delivery System, a CO-2060 plus Intelligent Column Thermostat/Selector, an HC-2068-01 Heater Controller, a BP-2080 plus Automatic Back Pressure Regulator, an MD-2018 plus Photodiode Array Detector (200-648 nm), and PU-2080 plus Intelligent HPLC Pumps. The purity of new compounds was judged by NMR and LCMS (>95%).

### Synthesis of Indoline CD4mc Intermediates

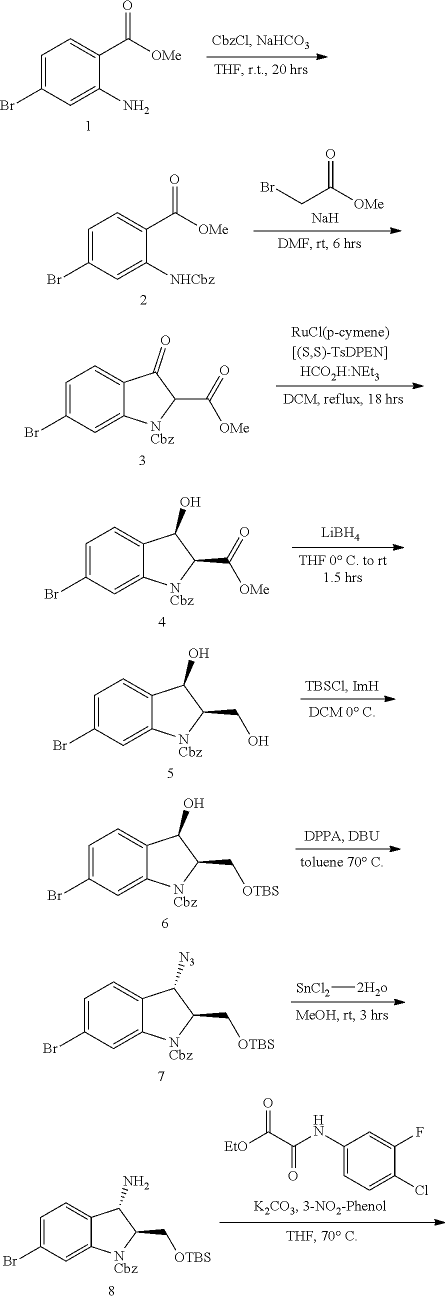

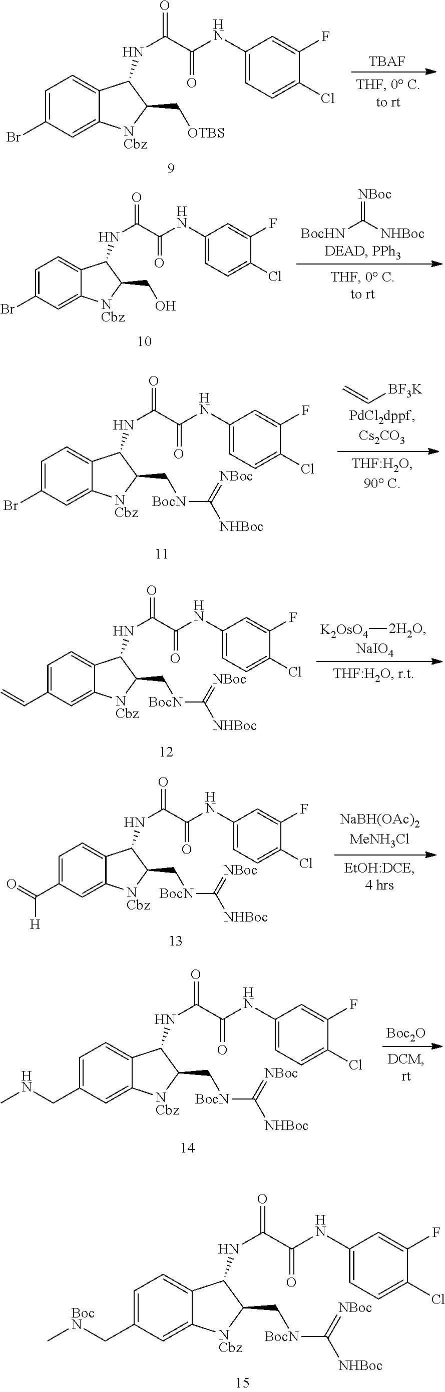

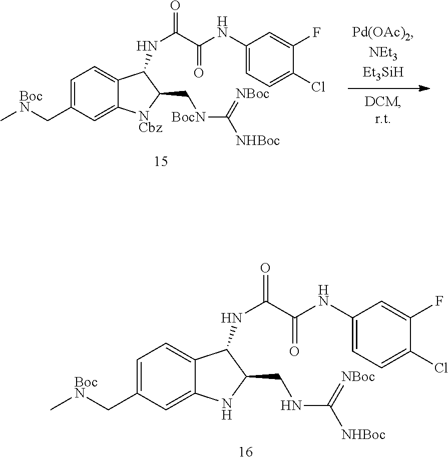

Methyl 2-(((benzyloxy)carbonyl)amino)-4-bromobenzoate (2) To a 3-neck 1 L round-bottomed flask fitted with a 50 mL addition funnel and magnetic stirring bar was added methyl 2-amino-4-bromobenzoate (1) (50.4 g, 219.1 mmol, 1.0 equiv.). The atmosphere was then purged and placed under argon. THE (438.2 mL, 0.50 M) was then added at room temperature, followed by NaHCO₃ (55.2 g, 657.4 mmol, 3.0 equiv.) under a positive pressure of argon. Benzyl chloroformate (46.72 mL, 328.7, 1.5 equiv.) was then added to the addition funnel and added dropwise to the heterogenous solution at a drop rate of approximately 1 drop/second. After completion of the addition, the reaction was allowed to stir at room temperature overnight. Distilled water (250 mL) was then added to the reaction mixture, and the aqueous layer was extracted with CH₂Cl₂ (3×250 mL). The organic layers were then combined, washed with brine, dried over Na₂SO₄, and concentrated in vacuo to a yellow solid. The solid was then triturated with 500 mL of a 1:3 mixture of CH₂Cl₂:hexanes and collected via vacuum filtration to obtain a yellow solid (2). The solvent of the filtrate was then concentrated in vacuo and resubjected to the same trituration conditions as described above to obtain a second crop of 2 (70.1 g, 88% yield).

¹H NMR (500 MHz, CDCl3) δ 10.59 (s, 1H), 8.72 (d, J=1.9 Hz, 1H), 7.85 (d, J=8.5 Hz, 1H), 7.45-7.41 (m, 2H), 7.40-7.36 (m, 2H), 7.36-7.32 (m, 1H), 7.16 (dd, J=8.5, 2.0 Hz, 1H), 5.22 (s, 2H), 3.90 (s, 3H); ¹³C NMR (125 MHz, CDCl3) δ 168.20, 153.37, 142.76, 136.10, 132.15, 129.87, 128.78, 128.53, 128.48, 125.10, 121.99, 113.40, 67.34, 52.62; HRMS (EI) m/z 363.0109 [calcd for C₁₆H₁₄BrNO₄ (M)⁺363.0106]. [0064]1-benzyl 2-methyl 6-bromo-3-oxoindoline-1,2-dicarboxylate (3) In an open 1 L round bottom flask with oversized magnetic stirring bar (or overhead stirring apparatus depending on scale), 2 (70.1 g, 192.5 mmol, 1.0 equiv.) was dissolved in DMF (385.0 mL, 0.5 M). To this solution was added methyl bromoacetate (19.1 mL, 202.1 mmol, 1.05 equiv.) at room temperature, followed by cesium carbonate (188.1 g, 577.43 mmol, 3.0 equiv.). The heterogenous mixture was then stirred at room temperature for 6 hours, at which time UPLCMS analysis indicated consumption of starting material. The remaining solid was filtered, and the filtrate was diluted with H₂O (400 mL). The mixture was then extracted with EtOAc (3×250 mL) and the organic layers were then combined, washed with brine (2×250 mL), dried over Na₂SO₄, and concentrated in vacuo. The product was precipitated by adding Et₂O and collected via vacuum filtration. The crude product was purified by flash column chromatography (20% EtOAc in hexanes) to give the product 3 as an off-white solid (66.1 g, 85% yield).

¹H NMR (500 MHz, CD₃OD) δ 8.50 (brs, 0.72H), 8.04 (brs, 0.14H), 7.60 (d, J=8.2 Hz, 1H), 7.43-7.35 (m, 6H), 5.40 (d, J=12.0 Hz, 1H), 5.15 (d, J=12.0 Hz, 1H), 3.60 (brs, 3H); ¹³C NMR (125 MHz, CD₃OD) δ ¹³C NMR (126 MHz, MeOD) δ 190.19, 179.46, 166.40, 155.46, 152.16, 136.82, 134.04, 129.85, 129.62, 128.60, 126.77, 123.35, 120.65, 69.64, 53.87, 49.77; HRMS (ESI) m/z 404.0139 [calcd for C₁₈H₁₅BrNO₅ (M+H)⁺ 404.0134].

1-benzyl 2-methyl (2S,3R)-6-bromo-3-hydroxyindoline-1,2-dicarboxylate (4) In a 2 L 2-neck round bottom flask fitted with a reflux condenser and magnetic stirring bar, 3 (66.1 g, 173.4 mmol, 1.0 equiv.) was added and capped with septa.

To the flask was then added freshly distilled and sparged (30 minutes with N₂ balloon) CH₂Cl₂ (694 mL, 0.25 M), followed by addition of RuCl[(S,S)-TsDPEN](p-cymene) (1.10 g, 1.73 mmol, 1 mol %). NEt₃ (31.4 mL, 225.5 mmol, 1.3 equiv.) was then added in one portion, followed by HCO₂H (20.9 mL, 555.0 mmol, 3.2 equiv.) dropwise via syringe. The reaction was then heated to reflux and stirred for 16 hours. Upon completion by TLC (30% EtOAc in hexanes), the reaction was quenched with water (500 mL), and the aqueous layer was extracted with CH₂Cl₂ (3×300 mL). The organic layers were combined, dried over Na₂SO₄, and concentrated in vacuo to a black oil. To the oil was added a 1:1 ratio of Et₂O:CH₂Cl₂ (400 mL) which incited precipitation. The solution was heated until full dissolution was observed. The solvent was allowed to evaporate slowly, forming crystals of 4 that were collected via vacuum filtration. ¹H NMR confirmed the d.r. to be >20:1 (57.8 g, 82% yield).

¹H NMR (500 MHz, CD₃OD) δ 8.07 (brs, 0.77H), 7.67 (brs, 0.16H), 7.46-7.33 (m, 5H), 7.26-7.18 (m, 2H), 5.55 (d, J=9.1 Hz, 1H), 5.31 (d, J=12.2 Hz, 1H), 5.10 (d, J=12.3 Hz, 1H), 5.01 (d, J=9.1 Hz, 1H), 3.58 (brs, 3H); ¹³C NMR (150 MHz, CD₃OD) δ 170.32, 153.63, 144.91, 137.29, 131.99, 129.75, 129.58, 129.38, 127.93, 127.46, 124.36, 118.70, 71.50, 68.91, 68.42, 52.65, 49.72; HRMS (ESI) m/z 406.0301 [calcd for C₁₈H₁₇BrNO₅ (M+H)⁺406.0290]; [α]_{D}²⁴-96.2 (c 0.76, MeOH).

Benzyl (2R,3R)-6-bromo-3-hydroxy-2-(hydroxymethyl)indoline-1-carboxylate (5) In a 2 L round-bottomed flask with magnetic stirring bar, 4 (57.8 g, 142.3 mmol, 1.0 equiv.) was added and dissolved in anhydrous THE (569.1 mL, 0.25 M). The solution was then cooled to 0° C. in an ice/water bath. LiBH4 powder (3.87 g, 177.9 mmol, 1.25 equiv.) was added to the reaction flask in 3 portions. The reaction was stirred at 0° C. for 30 minutes and warmed to room temperature. Upon equilibration and stirring for an additional hour, TLC (50% EtOAc in hexanes) indicated complete consumption of starting material. The reaction was cooled to 0° C. quenched with the addition of distilled water (300 mL). The reaction was then warmed to room temperature and the aqueous layer was extracted with Et₂O (3×250 mL). The organic layer was dried over Na₂SO₄, filtered, and concentrated in vacuo. The crude product was purified by flash column chromatography (40% EtOAc/hexanes) to give 5 as a white solid (36.1 g, 67%).

¹H NMR (500 MHz, CD₃OD) δ 7.88 (brs, 1H), 7.45 (d, J=7.0 Hz, 2H), 7.39 (t, J=7.2 Hz, 2H), 7.36-7.32 (m, 1H), 7.21 (d, J=7.9 Hz, 1H), 7.17 (dd, J=7.9, 1.7 Hz, 1H), 5.52 (d, J=8.8 Hz, 1H), 5.32-5.25 (m, 2H), 4.45 (ddd, J=8.4, 4.6, 3.2 Hz, 1H), 4.00 (dd, J=11.8, 4.7 Hz, 1H), 3.96 (dd, J=11.8, 3.3 Hz, 1H); ¹³C NMR (125 MHz, CD₃OD) δ 154.60, 137.57, 134.44, 129.84, 129.59, 129.52, 127.29, 127.04, 123.46, 119.43, 72.38, 68.96, 66.18, 60.72, 49.78; HRMS (ESI) m/z 400.0160 [calcd for C₁₇H₁₆BrNO₄Na (M+Na)⁺400.0160]; [α]_{D}²³ −22.7 (c 1.00, MeOH).

Benzyl (2R,3R)-6-bromo-2-(((tert-butyldimethylsilyl)oxy)methyl)-3-hydroxyindoline-1-carboxylate (6) To a suspension of 5 (36.1 g, 95.4 mmol, 1.0 equiv.) in CH₂Cl₂ (191 mL, 0.5 M) at 0° C. was added imidazole (13.0 g, 191.0 mmol, 2.0 equiv.) in one portion and stirred for 10 min. To this mixture was added a solution of tert-butyldimethylsilyl chloride (15.8 g, 105.0 mmol, 1.1 equiv.) in CH₂Cl₂ (105 mL, 1.0 M) dropwise over 30 min via dropping funnel. The reaction mixture was stirred at 0° C. for 30 min. Upon consumption of starting material based on TLC, the resulting mixture was treated with H₂O (150 mL). The aqueous phase was then extracted with CH₂Cl₂ (3×100 mL), the organic layers were dried over Na₂SO₄, filtered, and concentrated in vacuo. The resulting oil was further purified by flash column chromatography (10% EtOAc/hexanes) to give 6 as a clear oil (43.7 g, 93%).

¹H NMR (500 MHz, CD₃OD) δ 7.83 (s, 1H), 7.45 (d, J=6.8 Hz, 2H), 7.41-7.33 (m, 3H), 7.19-7.15 (d, J=7.9 Hz, 1H), 7.13 (d, J=8.0 Hz, 1H), 5.48 (d, J=8.8 Hz, 1H), 5.32 (d, J=12.0 Hz, 1H), 5.22 (d, J=12.2 Hz, 1H), 4.47 (dtd, J=8.8, 2.9, 1.3 Hz, 1H), 4.06 (d, J=2.8 Hz, 2H), 0.63 (s, 9H), −0.11 (s, 3H), −0.22 (s, 3H); ¹³C NMR (125 MHz, CD₃OD) δ 154.32, 144.68, 137.52, 135.31, 129.88, 129.77, 129.69, 127.13, 126.31, 123.13, 119.10, 72.60, 68.85, 66.17, 61.72, 26.11, 18.79, −5.46, −5.49; HRMS (ESI) m/z 492.1211 [calcd for C₂₃H₃₁BrNO₄Si (M+H)⁺492.1206]; [α]_{D}²³+48.3 (c 1.00, MeOH).

Benzyl (2S,3S)-3-azido-6-bromo-2-(((tert-butyldimethylsilyl)oxy)methyl)indoline-1-carboxylate (7) To a solution of 6 (43.7 g, 88.7 mmol, 1.0 equiv.) in toluene (296.0 mL, 0.3 M) was added diphenylphosphoryl azide (28.3 mL, 131.3 mmol, 1.48 equiv.) dropwise for 10 min, followed by dropwise addition of DBU (19.1 mL, 127.8 mmol, 1.44 equiv.) over 10 min. A cloudy mixture was observed upon addition, which was stirred at room temperature overnight. The resulting biphasic red mixture filtered through a pad of silica and washed with Et₂O (500 mL). The filtrate was concentrated in vacuo, and the resulting oil was further purified by flash column chromatography (2% Et₂O/hexanes) to give 7 as a clear oil (26.2 g, 57%).

¹H NMR (500 MHz, CD₃OD) δ 8.04 (brs, 0.72H), 7.66 (brs, 0.20H), 7.46 (d, J=6.8 Hz, 2H), 7.42-7.34 (m, 3H), 7.32 (d, J=8.0 Hz, 1H), 7.23 (d, J=8.0, Hz, 1H), 5.38 (brs, 1H), 5.17 (brs, 1H), 4.89 (s, 1H), 4.30 (s, 1H), 3.85 (d, J=10.1 Hz, 1H), 3.68 (brs, 1H), 0.67 (s, 9H), −0.11 (brs, 3H), −0.20 (brs, 3H); ¹³C NMR (150 MHz, CD₃OD) δ 153.58, 145.89, 137.32, 129.95, 129.86, 129.83, 128.77, 127.96, 127.36, 125.04, 119.53, 69.31, 69.02, 63.90, 60.27, 26.11, 18.87, −5.41, −5.51; HRMS (ESI) m/z 517.1282 [calcd for C₂₃H₃₀BrN₄O₃Si (M+H)⁺ 517.1271]; [α]_{D}²³+49.0 (c 1.00, MeOH).

Benzyl (2S,3S)-3-amino-6-bromo-2-(((tert-butyldimethylsilyl)oxy)methyl)indoline-1-carboxylate (8) To a solution 7 (26.2 g, 50.6 mmol, 1.0 equiv.) in MeOH (92.1 mL, 0.55 M) at room temperature, was added SnCl₂·2H₂O (17.1 g, 75.9 mmol, 1.5 equiv.) in a solution of MeOH (152 mL, 0.5 M). The solution was stirred for 3 hours at room temperature. Upon completion by TLC, 1M NaOH (100 mL) was added and the resulting precipitate was filtered through a thick pad of Celite®. The Celite @pad was washed with copious MeOH (200 mL) and the filtrate was concentrated in vacuo. The resulting oil was further purified by flash column chromatography (5% MeOH/CH₂Cl₂) to give 8 as a clear oil (17.9 g, 72%).

¹H NMR (600 MHz, CD₃OD) δ 7.99 (brs, 0.70H), 7.62 (s, 0.21H), 7.46 (d, J=6.9 Hz, 2H), 7.41-7.33 (m, 3H), 7.27-7.24 (m, 1H), 7.14 (d, J=7.6 Hz, 1H), 5.35 (d, J=11.6 Hz, 1H), 5.18 (brs, 1H), 4.25 (d, J=1.9 Hz, 1H), 4.14 (td, J=3.4, 1.9 Hz, 1H), 4.04-3.71 (br m, 2H), 0.65 (s, 9H), −0.09 (brs, 3H), −0.20 (brs, 3H); ¹³C NMR (150 MHz, CD₃OD) δ 154.20, 145.69, 137.51, 130.87, 129.88, 129.80, 129.70, 127.07, 126.24, 123.22, 119.23, 71.74, 68.73, 64.49, 55.61, 26.13, 18.88, −5.40, −5.46; HRMS (ESI) m/z 474.1106 [calcd for C₂₃H₂₉BrNO₃Si (M+H−NH₃)⁺ 474.1100]; [α]_{D}²³+16.0 (c 1.00, MeOH).

Benzyl (2S,3S)-6-bromo-2-(((tert-butyldimethylsilyl)oxy)methyl)-3-(2-((4-chloro-3-fluoro phenyl)amino)-2-oxoacetamido)indoline-1-carboxylate (10) A mixture of 8 (17.9 g, 36.4 mmol, 1.0 equiv.), 9 (11.2 g, 45.5 mmol, 1.25 equiv.), 3-nitrophenol (1.00 g, 7.28 mmol, 0.2 equiv.), and K₂CO₃ (1.01 g, 7.28 mmol, 0.2 equiv.) in THF (72.8 mL, 0.5 M) was stirred at reflux for 48 h. The resulting suspension was allowed to cool to room temperature and then treated with 10 wt % K₂CO₃ (50 mL) and stirred at room temperature for 1 hr. The mixture was then diluted with EtOAc (100 mL). The insoluble material was removed via vacuum filtration, and the solid was rinsed with EtOAc (2×100 mL). After, the filtrates were combined, the organic layer was separated and washed with 10 wt % K₂CO₃ (50 mL). The aqueous layers were combined and extracted with EtOAc (2×50 mL). The organic layers were combined and washed with brine, dried over Na₂SO₄, filtered, and concentrated in vacuo to give crude 10 as a brown oil. The resulting oil was further purified by flash column chromatography (20% EtOAc/hexanes) to give 10 as a yellow solid (9.3 g, 37%).

¹H NMR (600 MHz, CD₃OD) δ 8.01 (s, 1H), 7.81 (dd, J=11.4, 2.4 Hz, 1H), 7.48-7.44 (m, 3H), 7.42-7.33 (m, 4H), 7.21 (d, J=8.0 Hz, 1H), 7.14 (d, J=7.6 Hz, 1H), 5.41 (d, J=2.4 Hz, 1H), 5.36 (brs, 1H), 5.16 (brs, 1H), 4.37-4.35 (m, 1H), 4.08-3.90 (br m, 2H), 0.65 (s, 9H), −0.09 (brs, 3H), −0.21 (brs, 3H); ¹³C NMR (150 MHz, CDCl₃) δ 161.08, 159.79, 159.24 (d, J_{CF}=246.0 Hz), 139.18 (d, J_{CF}=9.4 Hz), 137.42, 131.79, 129.90, 129.85, 129.75, 127.71, 127.30, 124.01, 119.32, 118.20 (d, J_{CF}=3.5 Hz), 117.29 (d, J_{CF} 18.1 Hz), 109.89 (d, J_{CF}=26.1 Hz), 69.31, 68.84, 64.72, 53.89, 26.10, 18.85, −5.37, −5.44; HRMS (ESI) m/z 690.1206 [calcd for C₃₁H₃₅BrClFN₃O₅Si (M+H)⁺ 690.1202]; [α]_{D}²⁴+87.4 (c 0.97, MeOH).

Benzyl (2S,3S)-6-bromo-2-(((tert-butyldimethylsilyl)oxy)methyl)-3-(2-((4-chloro-3-fluoro phenyl)amino)-2-oxoacetamido)indoline-1-carboxylate compound with benzyl (2S,3S)-6-bromo-3-(2-((4-chloro-3-fluorophenyl)amino)-2-oxoacetamido)-2-(hydroxymethyl)indoline-1-carboxylate (11) Compound 10 (9.3 g, 13.5 mmol, 1.0 equiv.) was dissolved in THE (40.4 mL), and the resulting solution was diluted with MeOH (13.5 mL) and H2O (13.5 mL), to which conc. HCl (4.86 mL, 0.36 mL/mmol) was added dropwise. The reaction mixture was stirred at room temperature overnight. The mixture was then treated with sat. NaHCO₃ (50 mL) and Et₂O (100 mL). The aqueous layer was further extracted with Et₂O (2×100 mL). The organic layers were combined, dried over Na₂SO₄, filtered, and concentrated in vacuo to give crude 11 as an off-white powder, which could be used in the next step without further purification (7.37 g, 95% yield).

¹H NMR (500 MHz, CD₃OD) δ 8.02 (s, 1H), 7.82 (dd, J=11.4, 2.3 Hz, 1H), 7.49-7.44 (m, 3H), 7.42-7.37 (m, 2H), 7.36-7.33 (m, 1H), 7.24 (d, J=8.1 Hz, 1H), 7.18 (d, J=7.9 Hz, 1H), 5.42 (d, J=2.7 Hz, 1H), 5.33 (d, J=11.9 Hz, 1H), 5.28 (brs, 1H), 4.38-4.36 (m, 1H), 3.87-3.73 (m, 1H); ¹³C NMR (150 MHz, CD₃OD) δ 161.13, 159.84, 159.24 (d, J_{CF}=244.9 Hz), 139.25 (d, J_{CF}=9.4 Hz), 137.48, 131.80, 130.88, 129.86, 129.63, 129.57, 128.01, 127.45, 124.11, 118.20 (d, J_{CF}=3.5 Hz), 117.27 (d, J_{CF}=18.1 Hz), 109.85 (d, J_{CF}=26.1 Hz), 69.43, 68.95, 63.09, 49.72; HRMS (ESI) m/z 576.0348 [calcd for C₂₅H₂₁BrClFN₃O₅ (M+H)⁺ 576.0337]; [α]_{D}²⁴+91.7 (c 0.22, MeOH).

Benzyl (2R,3S)-6-bromo-3-(2-((4-chloro-3-fluorophenyl)amino)-2-oxoacetamido)-2-(((Z)-1,2,3-tris(tert-butoxycarbonyl)guanidino)methyl)indoline-1-carboxylate (12) In a 1 L round-bottomed flask with magnetic stirring bar, a mixture of 11 (7.37 g, 12.8 mmol, 1.0 equiv.), PPh₃ (5.36 g, 20.4 mmol, 1.6 equiv.), and N,N′,N″-tri-Boc-guanidine (16.1 g, 44.7 mmol, 3.5 equiv.) in anhydrous THE (320 mL, 0.04 M) was stirred at room temperature until a well-dispersed suspension had formed. This mixture was cooled at 0° C. and treated with diethyl azodicarboxylate (3.01 mL, 19.2 mmol, 1.5 equiv.) dropwise at such a rate that each drop was only added after the color change resulting from the previous drop had dissipated. After the addition was completed, the reaction was allowed to warm to room temperature and stir for 18 hr. TLC (30% EtOAc/hexanes) indicated complete consumption of starting material at this time. The reaction was then quenched with brine (100 mL) and the aqueous layer was extracted with EtOAc (3×100 mL). The organic layers were combined, dried over Na₂SO₄, and concentrated in vacuo to give a white solid which was purified by flash column chromatography (20% EtOAc/hexanes) to give a mixture of 12 and N,N′,N″-tri-Boc-guanidine. The residue was treated with Et₂O (20 mL) and the insoluble material was removed via vacuum filtration. The filtrate was concentrated in vacuo to give 12 as an off-white amorphous solid (7.63 g, 65%).

¹H NMR (500 MHz, CD₃OD) δ 7.99 (brs, 1H), 7.81 (dd, J=11.4, 2.3 Hz, 1H), 7.50 (d, J=7.1 Hz, 2H), 7.48-7.41 (m, 2H), 7.40-7.29 (m, 3H), 7.27 (d, J=8.0 Hz, 1H), 7.20 (dd, J=8.0, 1.8 Hz, 1H), 5.37-5.25 (br m, 3H), 4.72 (s, 1H), 4.15 (dd, J=14.2, 6.3 Hz, 1H), 4.02 (s, 1H), 1.54-1.42 (m, 27H); ¹³C NMR (150 MHz, CD₃OD) δ 161.12, 159.70, 159.24 (d, J_{CF}=246.0 Hz), 155.17, 151.51, 139.24, 139.18, 137.54, 131.81, 130.01, 129.79, 129.72, 129.54, 128.58, 127.79, 124.34, 118.18 (d, J_{CF}=3.5 Hz), 117.28 (d, J_{CF}=18.1 Hz), 109.84 (d, J_{CF}=26.1 Hz), 85.76, 83.72, 67.04, 28.62, 28.60, 28.41; HRMS (ESI) m/z 917.2286 [calcd for C₄₁H₄₈BrClFN₆O₁₀ (M+H)⁺ 917.2288]; [α]_{D}²⁴+54.9 (c 0.77, MeOH).

Benzyl (2R,3S)-3-(2-((4-chloro-3-fluorophenyl)amino)-2-oxoacetamido)-2-(((Z)-1,2,3-tris (tert-butoxycarbonyl)guanidino)methyl)-6-vinylindoline-1-carboxylate (13) A flame dried flask equipped with magnetic stirring bar was charged with 12 (7.63 g, 8.31 mmol, 1.0 equiv.), potassium vinyltrifluoroborate (1.14 g, 9.97 mmol, 1.2 equiv.), [1,1′-Bis(diphenyl-phosphino)ferrocene]palladium(II) dichloride (339 mg, 0.42 mmol, 5 mol %) and cesium carbonate (8.12 g, 24.9 mmol, 3.0 equiv.). The flask was evacuated and backfilled with N₂ atmosphere 3 times before the addition of freshly distilled THE (66.5 mL) and distilled H₂O (16.7 mL). The reaction flask was then evacuated and backfilled 4 times before heating to reflux. The reaction mixture was stirred at reflux for 18 hr, at which time UPLCMS analysis indicated full consumption of starting material. The reaction was then cooled to room temperature, diluted with Et₂O and H₂O (100 mL each). The aqueous phase was extracted with Et₂O (2×100 mL), and the organic layers were combined, dried over Na₂SO₄, filtered, and concentrated in vacuo to yield crude 13. The crude solid was further purified by flash column chromatography (20% EtOAc/hexanes) to give 13 as a white amorphous solid (5.11 g, 71%).

¹H NMR (500 MHz, CD₃OD) δ 7.93 (s, 1H), 7.81 (dd, J=11.4, 2.3 Hz, 1H), 7.51 (d, J=7.5 Hz, 2H), 7.46 (dd, J=9.3, 2.1 Hz, 1H), 7.42 (d, J=8.2 Hz, 1H), 7.41-7.33 (m, 1H), 7.31 (d, J=7.8 Hz, 1H), 7.12 (brs, 1H), 6.71 (brs, 1H), 5.64 (brs, 1H), 5.37-5.19 (br m, 2H), 4.74 (s, 1H), 4.16 (dd, J=14.1, 6.4 Hz, 1H), 3.97 (dd, J=14.2, 7.3 Hz, 1H), 1.48-1.43 (m, 27H); ¹³C NMR (150 MHz, CD₃OD) δ 161.09, 159.76, 159.24 (d, J_{CF}=246.0 Hz), 155.26, 140.98, 139.21 (d, J_{CF}=9.4 Hz), 138.20, 132.55, 131.81, 130.02, 129.78, 129.72, 129.51, 127.26, 123.63, 118.18 (d, J_{CF}=3.5 Hz), 117.28 (d, J_{CF}=18.1 Hz), 114.98, 109.86 (d, J_{CF}=26.1 Hz), 85.75, 83.69, 66.91, 28.61, 28.42; HRMS (ESI) m/z 865.3354 [calcd for C₄₃H₅₁ClFN₆O₁₀ (M+H)⁺ 865.3339]; [α]_{D}²⁴+68.4 (c 0.60, MeOH).

Benzyl (2R,3S)-3-(2-((4-chloro-3-fluorophenyl)amino)-2-oxoacetamido)-6-formyl-2-(((Z)p-1,2,3-tris(tert-butoxycarbonyl)guanidino)methyl)indoline-1-carboxylate (14) To a solution of 13 (5.11 g, 8.82 mmol, 1.0 equiv.) in a 4:1 mixture of THF/H₂O (44.1 mL, 0.2 M), was added 2,6-lutidine (4.09 mL, 35.3 mmol, 4.0 equiv.) dropwise via syringe, followed by potassium osmate dihydrate (65 mg, 0.18 mmol, 2 mol %) in one portion, and sodium periodate (5.66 g, 26.5 mmol, 3.0 equiv.) in one portion. The cloudy white reaction slurry was stirred for 2 hr at room temperature until completion via TLC. The reaction was quenched by the addition of sat. NaHCO₃ (50 mL), diluted with Et₂O (50 mL). The aqueous phase was extracted with Et₂O (2×50 mL), and the organic layers were combined, dried over Na₂SO₄, filtered, and concentrated in vacuo to yield crude 14. The crude solid was further purified by flash column chromatography (25% EtOAc/hexanes) to give 14 as a white amorphous solid (4.51 g, 59%).

¹H NMR (500 MHz, (CD₃)₂CO) δ 10.52 (brs, 0.5H), 10.12 (s, 0.5H), 10.02 (brs, 1H), 9.17 (d, J=8.3 Hz, 1H), 8.31 (brs, 1H), 7.98-7.93 (m, 1H), 7.71-7.69 (m, 1H), 7.62-7.57 (m, 4H), 7.51 (t, J=8.6 Hz, 1H), 7.40 (t, J=7.4 Hz, 2H), 7.35 (t, J=7.3 Hz, 1H), 5.69 (brs, 1H), 5.37 (d, J=12.1 Hz, 1H), 5.33-5.28 (br m, 1H), 4.87 (brs, 1H), 4.27 (dd, J=13.9, 5.2 Hz, 1H), 4.23 (brs, 1H), 1.50-1.39 (m, 27H); ¹³C NMR (150 MHz, (CD₃)₂CO) δ 192.65, 170.98, 160.09, 159.07, 158.59 (d, J_{CF}=246.0 Hz), 154.23, 139.03 (d, J_{CF}=10.2 Hz), 137.33, 131.63, 129.41, 129.08, 126.41, 117.88 (d, J_{CF}=3.5 Hz), 116.32 (d, J_{CF}=17.7 Hz), 109.24 (d, J_{CF}=25.2 Hz), 84.55, 66.11, 60.61, 48.53, 30.43, 28.36, 20.90, 14.58; HRMS (ESI) m/z 867.3136 [calcd for C₄₂H₄₉ClFN₆O₁₁ (M+H)⁺ 867.3132]; [α]_{D}²³+83.48 (c 1.00, MeOH).

Benzyl (2R,3S)-6-(((tert-butoxycarbonyl)(methyl)amino)methyl)-3-(2-((4-chloro-3-fluoro phenyl)amino)-2-oxoacetamido)-2-(((Z)-1,2,3-tris(tert-butoxycarbonyl)guanidino)methyl)-indoline-1-carboxylate (15) To a stirring solution of 14 (4.51 g, 5.20 mmol, 1.0 equiv.) in a 1:1 mixture of EtOH/DCE (52.0 mL, 0.1 M) at 0° C. was added methylamine hydrochloride (1.76 g, 26.0 mmol, 5.0 equiv.) in one portion. The reaction was stirred for 10 minutes at 0° C. before the addition of sodium triacetoxyborohydride (2.76 g, 13.0 mmol, 2.5 equiv.) in one portion. The ice bath was then removed, and the reaction mixture was allowed to warm to room temperature. The reaction was allowed to stir overnight, at which time complete consumption of 14 was observed by TLC and confirmed by UPLCMS. The reaction was then diluted with CH₂Cl₂ (50 mL), quenched with the slow addition of H₂O (50 mL). The aqueous phase was basified to a pH of 12 with aq. 1 N NaOH solution and extracted with CH₂Cl₂ (3×50 mL). The organic layers were combined, dried over Na₂SO₄, and concentrated in vacuo to give an oil. This was dissolved in CH₂Cl₂ (10.4 mL, 0.5M) and cooled to 0° C., to which Boc20 (1.43 mL, 6.24 mmol, 1.2 equiv) was added dropwise via syringe. The reaction mixture was warmed to room temperature and stirred for 1 hour. The solvent was then concentrated in vacuo to give crude 15 as an oil, which was purified by flash column chromatography (20% EtOAc/hexanes) to give the product 15 as an amorphous solid (3.58 g, 70% over 2 steps).

¹H NMR (500 MHz, CD₃OD) δ 7.81 (dd, J=11.4, 2.3 Hz, 1H), 7.79 (brs, 1H), 7.49 (d, J=7.1 Hz, 2H), 7.46 (dd, J=9.1, 2.3 Hz, 1H), 7.44-7.30 (m, 5H), 6.96 (dd, J=7.7, 1.5 Hz, 1H), 5.36-5.24 (m, 3H), 4.75-4.71 (br m, 1H), 4.42 (brs, 2H), 4.15 (dd, J=14.1, 6.0 Hz, 1H), 3.97 (dd, J=14.1, 7.6 Hz, 1H), 2.80 (brs, 3H), 1.49-1.44 (m, 36H); ¹³C NMR (150 MHz, CD₃OD) δ 161.07, 159.77, 159.24 (d, J_{CF}=246.0 Hz), 155.24, 139.22 (d, J_{CF}=9.4 Hz), 131.81, 129.74, 129.47, 124.17, 118.17 (d, J_{CF}=3.5 Hz), 117.27 (d, J_{CF}=18.1 Hz), 109.85 (d, J_{CF}=26.1 Hz), 85.71, 83.71, 73.41, 66.95, 49.72, 28.88, 28.63, 28.44, 22.07; HRMS (ESI) m/z 982.4113 [calcd for C₄₈H₆₂ClFN₇O₁₂ (M+H)⁺ 982.4129]; [α]_{D}²⁴+57.0 (c 0.85, MeOH).

### General Procedure for the Synthesis of Final Compounds

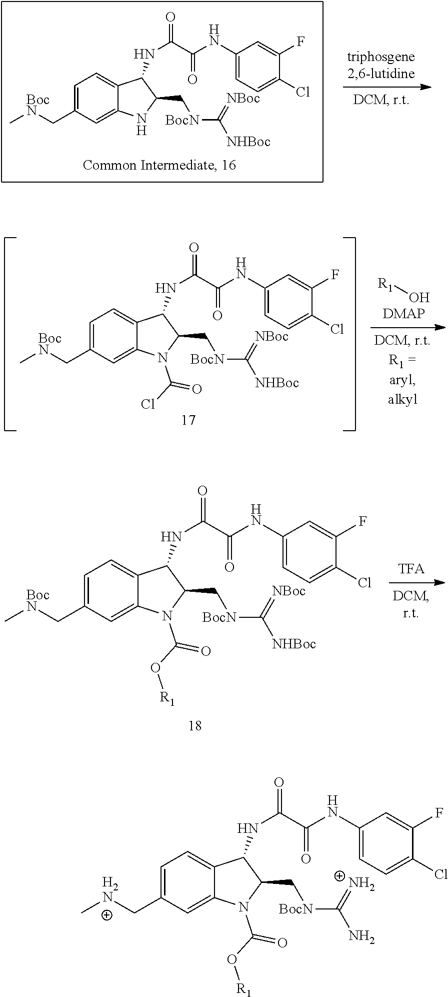

Carbamoyl Chloride (17) Compound 13 (500 mg, 0.51 mmol, 1.0 equiv.) was dissolved in CH₂Cl₂ (5.09 mL, 0.1 M) and cooled to 0° C. under argon. In a separate flask, Pd(OAc)₂ (8.0 mg, 0.036 mmol, 7 mol %) and NEt₃ (17.7 μL, 0.127 mmol, 0.25 equiv.) were dissolved in CH₂Cl₂ (1.02 mL). Et₃SiH (139.8 μL, 0.88 mmol, 1.72 equiv.) was then added in one portion to the mixture. Upon turning black, the solution was taken up via syringe and added to the solution of 15 dropwise. The reaction was allowed to warm to r.t. and stirred for 4 hours, at which time UPLCMS indicated complete consumption of starting material. Upon completion, excess NEt₃ (1 mL) was added and the solution was filtered through a pad of Celite®. This was washed with CH₂Cl₂ (5 mL) and concentrated in vacuo. The crude material was then immediately passed through a plug of silica pre-saturated with 20% EtOAc/hexanes with 1% NEt₃ and washed with the same mixture (50 mL) until elution of the desired product 16 was observed by TLC (351.9 mg).

This material was then redissolved in CH₂Cl₂ (2.07 mL), followed by addition of 2,6-lutidine (50.5 μL, 0.436 mmol, 1.05 equiv.). The mixture was then cooled to 0° C., and a 0.2M stock solution of triphosgene in CH₂Cl₂ (0.73 mL, 0.14 mmol, 0.35 equiv.) was added dropwise. The reaction was allowed to stir for 2 hours, at which time UPLCMS analysis indicated consumption of 16. The reaction was quenched with sat. aq. NH₄Cl (2 mL), and the aqueous layer was extracted 3×2 mL CH₂Cl₂. The organic layers were separated, dried with Na₂SO₄, filtered and concentrated in vacuo. The crude carbamoyl chloride 15 was then purified by flash column chromatography (20% EtOAc/hexanes) to give 17 as a red amorphous solid (374.4 mg, 81% over 2 steps).

¹H NMR (600 MHz, (CD₃)₂CO) δ 10.27 (s, 0.5H), 10.13 (s, 0.5H), 9.25 (d, J=8.2 Hz, 1H), 8.02-7.92 (m, 1H), 7.82 (brs, 1H), 7.70 (dt, J=8.9, 1.3 Hz, 1H), 7.51 (t, J=8.6 Hz, 1H), 7.47 (brs, 1H), 7.13 (brs, 1H), 5.56 (brs, 1H), 5.02-4.98 (m, 1H), 4.55-4.42 (m, 2H), 4.30 (dd, J=14.2, 5.2 Hz, 1H), 4.12-4.04 (br m, 1H), 2.79 (s, 3H, obscured by residual H₂O), 1.51 (brs, 9H), 1.48 (brs, 9H), 1.46-1.45 (br m, 18H); ¹³C NMR (150 MHz, (CD₃)₂CO) δ 160.06, 159.06, 158.59 (d, J_{CF}=244.9 Hz), 154.13, 145.68, 142.86, 141.79, 139.03 (d, J_{CF}=9.5 Hz), 131.64, 125.89, 117.90 (d, J_{CF}=3.5 Hz), 116.34 (d, J_{CF}=17.7 Hz), 109.24 (d, J_{CF}=25.2 Hz), 84.85, 69.38, 52.88, 52.78, 48.40, 30.42, 28.66, 28.35, 28.25, 23.36, 22.28, 22.01, 14.42; HIRMS (ESI) M/Z 910.3319 [CALCD FOR C₄₁H₅₅CL₂FN₇O₁₁ (M+H)⁺ 910.3321]; [α]_{D}²⁴+71.1 (c 0.52, MeOH).

Compound 18. Compound 17 was dissolved in CH₂Cl₂ (0.1 M), followed by addition of the alcohol desired for carbamate formation (2.0 equiv.). N,N-dimethylaminopyridine (1.5 equiv.) was then added in one portion, and the mixture was allowed to stir overnight, at which time UPLCMS analysis indicated consumption of 17. The reaction was quenched with sat. aq. NH₄Cl, and the aqueous layer was extracted 3× CH₂Cl₂. The organic layers were separated, dried with Na₂SO₄, filtered and concentrated in vacuo. The crude 18 (1.0 equiv.) was then taken up in CH₂Cl₂ (0.1 M) and cooled to 0° C. in an ice-water bath. Trifluoroacetic acid (40 equiv.) was added and the mixture was allowed to warm to rt. The reaction was allowed to stir for 18 hours. The solution was then concentrated in vacuo and the resulting crude residue was purified by flash column chromatography (10% MeOH/CH₂Cl₂) to give the products as an amorphous white solids.

### EXAMPLES

Compounds. Compounds of the disclosure were prepared according to the methods described herein.

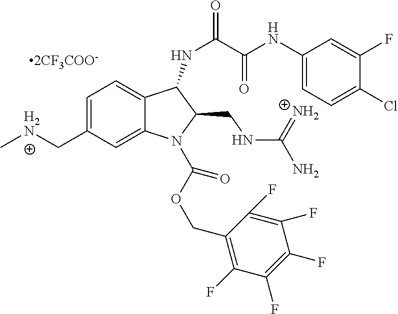

¹H NMR (500 MHz, MeOD) δ 7.98 (brs, 1H), 7.82 (dd, J=11.3, 2.2 Hz, 1H), 7.53-7.37 (m, 3H), 7.27-7.20 (m, 1H), 5.51 (brs, 2H), 5.23 (s, 1H), 4.58-4.49 (m, 1H), 4.21 (s, 2H), 3.54 (qd, J=14.1, 6.1 Hz, 2H), 2.72 (s, 3H).

LRMS (ESI) m/z 672.398 [calcd for C₂₈H₂₅ClF₆N₇O₄ (M+H)⁺ 672.1555].

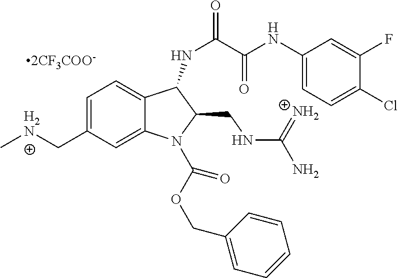

### CJF-III-192

¹H NMR (500 MHz, MeOD) δ 7.93 (brs, 1H), 7.82 (dd, J=11.3, 2.0 Hz, 1H), 7.51-7.33 (m, 8H), 7.22 (dd, J=7.8, 1.4 Hz, 1H), 5.37 (brs, 2H), 5.25 (d, J=2.2 Hz, 1H), 4.57 (ddd, J=7.4, 4.8, 2.5 Hz, 1H), 4.21 (s, 2H), 3.59 (dd, J=14.0, 4.9 Hz, 1H), 3.51 (dd, J=14.1, 7.3 Hz, 1H), 2.70 (s, 3H).

HRMS (ESI) m/z 582.2029 [calcd for C₂₈H₃₀ClFN₇O₄ (M+H)⁺ 582.2032].

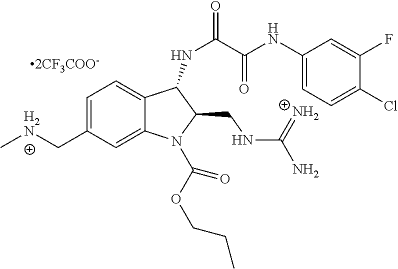

### CJF-III-288-B

¹H NMR (500 MHz, MeOD) δ 7.89 (brs, 1H), 7.83 (dd, J=11.3, 2.2 Hz, 1H), 7.53-7.40 (m, 3H), 7.22 (d, J=7.7 Hz, 1H), 5.24 (d, J=2.0 Hz, 1H), 4.57-4.54 (m, 1H), 4.32-4.19 (m, 4H), 3.60 (dd, J=14.1, 5.1 Hz, 1H), 3.54 (dd, J=14.0, 7.0 Hz, 1H), 2.73 (s, 3H), 1.81 (q, J=7.0 Hz, 2H), 1.03 (t, J=7.4 Hz, 3H).

HRMS (ESI) m/z 534.2027 [calcd for C₂₄H₃₀ClFN₇O₄ (M+H)⁺ 534.2032].

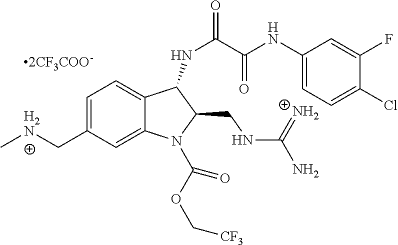

### CJF-IV-046-C

¹H NMR (500 MHz, MeOD) δ), 8.00 (brs, 1H), 7.83 (dd, J=11.3, 2.3 Hz, 1H), 7.53 (d, J=7.8 Hz, 1H), 7.50-7.41 (m, 2H), 7.27 (d, J=7.7 Hz, 1H), 5.28 (s, 1H), 4.86 (signal obscured by residual MeOH, 2H), 4.63-4.59 (m, 1H), 4.23 (s, 1H), 3.59 (qd, J=14.2, 6.2 Hz, 2H), 2.73 (s, 3H).

LRMS (ESI) m/z 574.325 [calcd for C₂₃H₂₅ClF₄N₇O₄ (M+H)⁺ 574.1587].

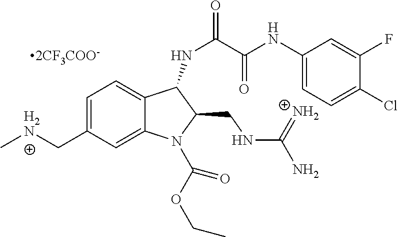

### CJF-III-288-C

¹H NMR (500 MHz, MeOD) δ 7.90 (s, 1H), 7.83 (dd, J=11.3, 2.1 Hz, 1H), 7.50-7.36 (m, 3H), 7.22 (d, J=7.8 Hz, 1H), 5.23 (s, 1H), 4.57-4.54 (m, 1H), 4.39-4.33 (m, 2H), 4.22 (s, 2H), 3.60-3.52 (m, 2H), 2.73 (s, 3H), 1.40 (t, J=7.0 Hz, 3H).

HRMS (ESI) m/z 520.1890 [calcd for C₂₃H₂₈ClFN₇O₄ (M+H)⁺ 520.1875].

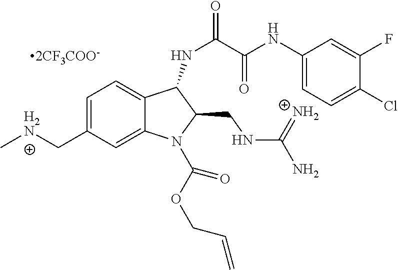

### CJF-III-280

¹H NMR (500 MHz, MeOD) δ 7.91 (brs, 1H), 7.83 (dd, J=11.3, 2.1 Hz, 2H), 7.54-7.37 (m, 3H), 7.23 (d, J=7.8 Hz, 2H), 6.13-6.00 (m, 1H), 5.42 (d, J=17.2 Hz, 1H), 5.31 (d, J=10.4 Hz, 1H), 5.24 (d, J=1.8 Hz, 1H), 4.59-4.56 (m, 1H), 4.22 (s, 2H), 3.61-3.52 (m, 2H), 2.73 (s, 3H).

HRMS (ESI) m/z 532.1870 [calcd for C₂₄H₂₈ClFN₇O₄ (M+H)⁺ 532.1875].

BNM-III-170 was prepared according to a previously described synthesis. Melillo, B.; et al., Small-Molecule CD4-Mimics: Structure-Based Optimization of HIV-1 Entry Inhibition. ACS Medicinal Chemistry Letters 2016, 7 (3), 330-334.

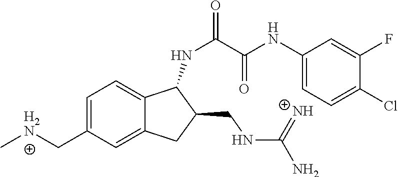

### BNM-III-170

Cell lines. 293T cells were grown in Dulbecco's modified Eagle's medium (DMEM) (Life Technologies, Wisent Inc.) supplemented with 10% fetal bovine serum (FBS) (Life Technologies, VWR) and 100 g/ml of penicillin-streptomycin (Life Technologies, Wisent Inc.). Cf2Th cells stably expressing the human CD4 and CCR5 coreceptors for HIV-1 were grown in the same medium supplemented with 0.4 mg/ml of G418 and 0.2 mg/ml of hygromycin.

CD4-mimetic compound. The compounds of the disclosure (CD4mc) were dissolved in dimethyl sulfoxide (DMSO) at a stock concentration of 10 mM and diluted to the appropriate concentration for antiviral assays.

Production of recombinant pseudoviruses expressing luciferase. 293T cells were transfected with pSVIIIenv plasmids expressing Env from HIV-1 strain variants, the pCMVΔP1Δenv HIV-1 Gag-Pol packaging construct and the firefly luciferase-expressing HIV-1 vector at a 1:1:3 μg DNA ratio using effectene transfection reagent (Qiagen). Recombinant, luciferase-expressing viruses capable of a single round of replication were released into the cell medium and were harvested 48 h later. The virus-containing supernatants were clarified by low-speed centrifugation (2000 rpm for 10 min) and used for single-round infections.

Virus infectivity and inhibition. Single-round virus infection assays were used to measure the ability of the Env variants to support virus entry. To measure the infectivity of the Env pseudotypes, recombinant viruses were added to Cf2Th target cells expressing CD4 and CCR5. Forty-eight h later, the target cells were lysed and the luciferase activity was measured.

For inhibition assays, the compounds to be tested were incubated at various concentrations with recombinant pseudoviruses for 1 h at 37° C. The mixture was then added to Cf2Th target cells expressing CD4 and CCR5. Forty-eight hours later, the target cells were lysed and the luciferase activity was measured.

Statistics. The concentrations of CD4-mimetic compounds that inhibit 50% of infection (IC50 values) were determined by fitting the data in five-parameter dose-response curves using GraphPad Prism 8.

### Results

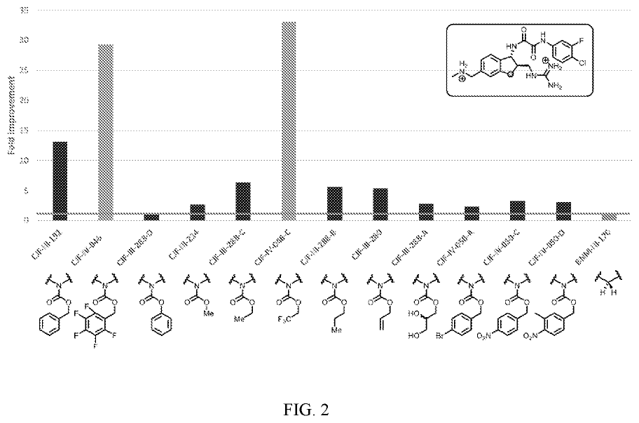

Compounds of the disclosure were tested for their ability to inhibit the viral entry process. CJF-III-192 demonstrated a 13-fold increase in potency and CJF-IV-046 resulted in a 30-fold improvement in viral inhibition compared to BNM-III-170. A summary of the normalized activity of all tested compounds is in FIG. 2.

Compounds of the disclosure were examined against BG505, a more difficult to neutralize strain of HIV-1. BG505 is a cladeA virus that has not been susceptible to viral entry inhibition by BNM-III-170 (>300 μM). Compounds of the disclosure displayed low-micromolar inhibition of BG505 entry into target cells. See Table 1.

TABLE 1

| | CJF-IV- | CJF-IV- | CJF- | CJF-III- | BNM- |
|---|---|---|---|---|---|
| | 046-B | 046-C | III-192 | 288-B | III-170 |
| AD8 | 0.1 | 0.1 | 0.13 | 0.1 | 1.7 |
| IC₅₀ (μM) | | | | | |
| JR-FL | 0.52 | 0.8 | 0.93 | 0.51 | 12.2 |
| IC₅₀ (μM) | | | | | |
| BG505 | 10.1 | 45.6 | 31.6 | 27.0 | >300 |
| IC₅₀ (μM) | | | | | |

CD₄mc resistant variants of Clade B virus AD8 were also selected by incubating infected host cells with increasing concentrations of BNM-III-170. After selection, mutant HIV-1AD8 batches 130_3 and 130_C were completely resistant to entry inhibition by BNM-III-170 up to 300 μM. These resistant strains of AD8 were sequenced to find specific mutations in the gp120 monomer that conferred the greatest resistance to BNM-III-170. Of these mutations examined, E64G, S375N, and I424T conferred the greatest resistance to treatment with compounds of the disclosure.

Each of the mutations individually were able to increase resistance to BNM-III-170, but were most resistant when combined as in HIV-1AD8 isolates 130_3 and 130_C. When exposed to CJF-III-192, viruses were neutralized to a greater degree than BNM-III-170. This was true with the individual mutations discussed above, as well as the original resistant mutants 130_3 and 130_C.

The ability of the compounds of the disclosure to enhance Ab recognition of Env on the surface of infected cells and mediate ADCC was examined. Via HIV+ plasma binding, it was observed that CJF-III-192 was able to enhance Ab recognition to a greater degree than BNM-III-170 (7-fold vs 2-fold enhancement vs DMSO) for JR-FL infected primary T-cells. Additionally, when comparing the % ADCC activity of these two compounds, it was observed that CJF-III-192 could achieve up to 8% killing of HIVJR-FL-infected cells, while BNM-III-170 could only attain levels up to 5%. This effect on ADCC activity was more pronounced with HIVCH58TF-infected cells; 18% killing for CJF-III-192 vs. 8% killing with BNM-III-140.

The compounds of the disclosure are improved in regard to (1) viral entry inhibition of JR-FL and other resistant HIV-1 strains, (2) host antibody recognition and CoRBS exposure of Env, and (3) killing of infected cells through ADCC.

Nothing in this specification should be considered as limiting the scope of this disclosure. All examples presented are representative and non-limiting. The above-described embodiments can be modified or varied, as appreciated by those skilled in the art in light of the above teachings. It is therefore to be understood that, within the scope of the claims and their equivalents, the embodiments disclosed herein can be practiced otherwise than as specifically described.

1. A compound of Formula I: 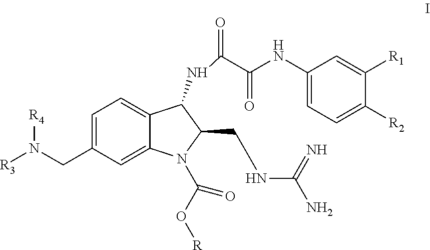
or stereoisomer thereof, wherein:
R is C₀₋₆alk-aryl, substituted C₀₋₆alk-aryl, C₁₋₆alkyl, substituted C₁₋₆alkyl, C₁₋₆haloalkyl, C₂₋₆alkenyl, or substituted C₂₋₆alkenyl;
R₁ is F, Cl, or Br;
R₂ is F, Cl, or Br;
R₃ is H or C₁₋₆alkyl; and
R₄ is H or C₁₋₆alkyl
or a pharmaceutically acceptable salt thereof.

2. The compound of claim 1, wherein R₁ is F or Cl.

3. The compound of claim 1, wherein R₂ is F or Cl.

4. The compound of claim 1, wherein R₁ is F and R₂ is Cl.

5. The compound of claim 1, wherein R₃ is H.

6. The compound of claim 1, wherein R₃ is C₁₋₆alkyl.

7. The compound of claim 1, wherein R₄ is C₁₋₆alkyl.

8. The compound of claim 1, wherein R₄ is H.

9. The compound of claim 1, wherein R₃ is C₁₋₆alkyl and R₄ is H.

10. The compound of claim 1, wherein R is C₀₋₆alk-aryl or substituted C₀₋₆alk-aryl.

11. The compound of claim 10, wherein R is C₁alkaryl.

12. The compound of claim 10, wherein R is substituted C₁alkaryl.

13. The compound of claim 10, wherein R is substituted C₁alkaryl.

14. The compound of claim 10, wherein R is phenyl.

15. The compound of claim 1, wherein R is C₁₋₆alkyl or substituted C₁₋₆alkyl.

16. The compound of claim 1, wherein R is methyl, ethyl, n-propyl, or 2,3-dihydroxpropyl.

17. The compound of claim 1, wherein R is C₁₋₆haloalkyl.

18. The compound of claim 1, wherein R is C₂₋₆alkenyl or substituted C₂₋₆alkenyl.

19. (canceled)

20. (canceled)

21. A pharmaceutical composition comprising a compound of claim 1 and a pharmaceutically acceptable excipient.

22. A method of treating or preventing HIV infection in a mammal comprising administering to the mammal a compound of claim 1.

23. A compound that is 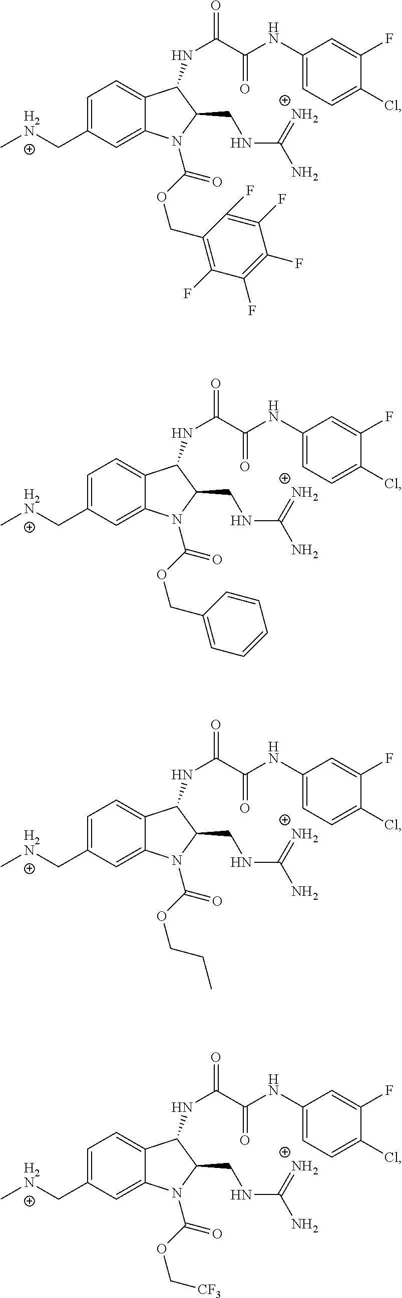 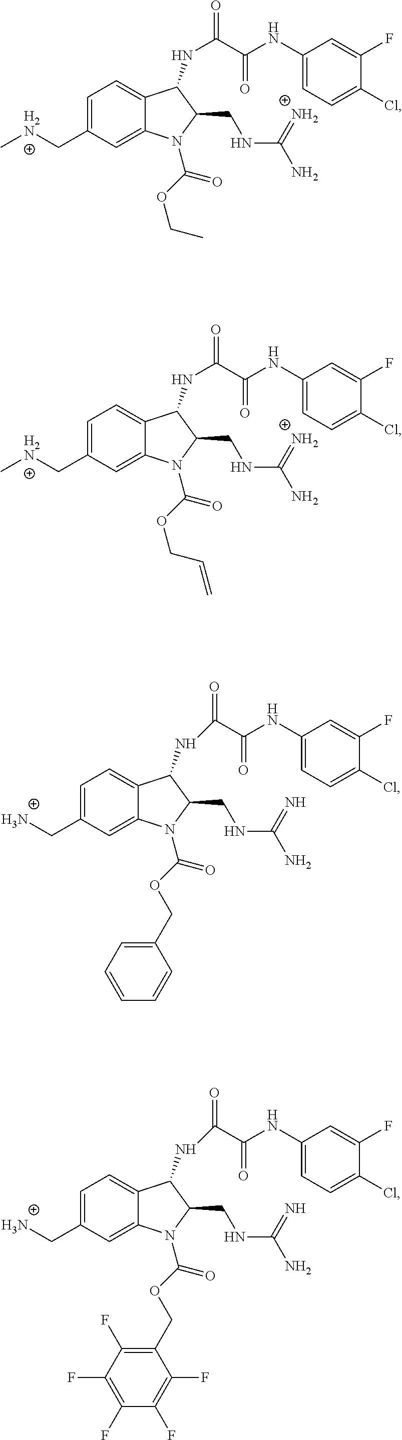 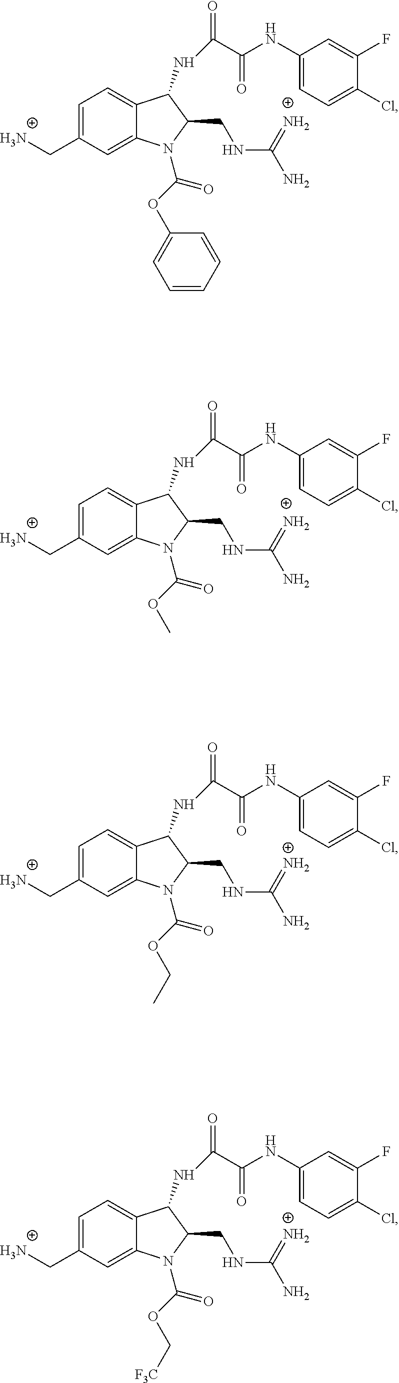 [SMILES: NC(=[NH2+])NC[C@@H]1[C@@H](NC(=O)C(=O)Nc2ccc(Cl)c(F)c2)c2ccc(C[NH3+])cc2N1C(=O)Oc1ccccc1 ; COC(=O)N1c2cc(C[NH3+])ccc2[C@H](NC(=O)C(=O)Nc2ccc(Cl)c(F)c2)[C@H]1CNC(N)=[NH2+] ; CCOC(=O)N1c2cc(C[NH3+])ccc2[C@H](NC(=O)C(=O)Nc2ccc(Cl)c(F)c2)[C@H]1CNC(N)=[NH2+] ; NC(=[NH2+])NC[C@@H]1[C@@H](NC(=O)C(=O)Nc2ccc(Cl)c(F)c2)c2ccc(C[NH3+])cc2N1C(=O)OCC(F)(F)F] 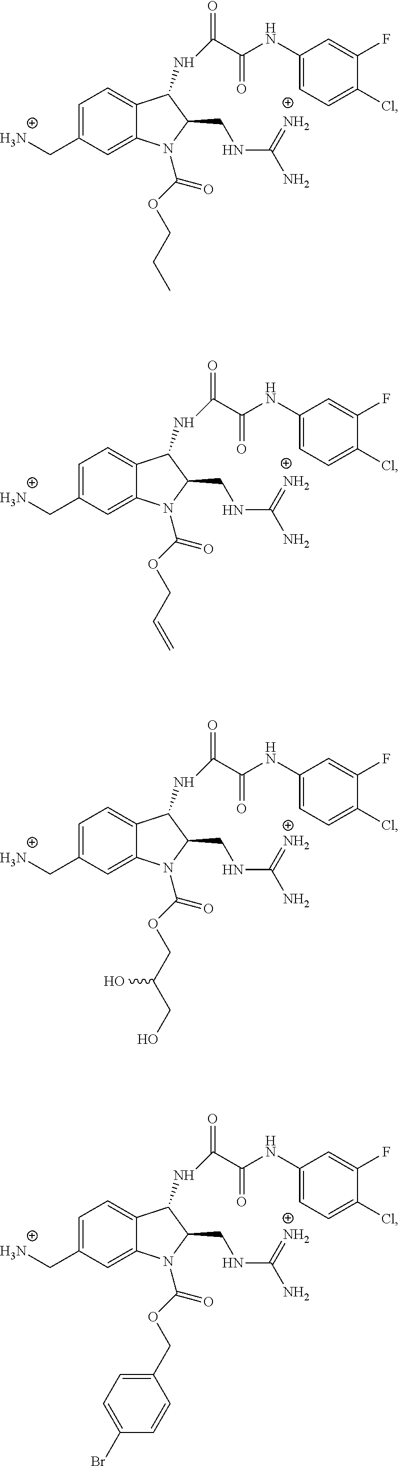 [SMILES: CCCOC(=O)N1c2cc(C[NH3+])ccc2[C@H](NC(=O)C(=O)Nc2ccc(Cl)c(F)c2)[C@H]1CNC(N)=[NH2+] ; C=CCOC(=O)N1c2cc(C[NH3+])ccc2[C@H](NC(=O)C(=O)Nc2ccc(Cl)c(F)c2)[C@H]1CNC(N)=[NH2+] ; NC(=[NH2+])NC[C@@H]1[C@@H](NC(=O)C(=O)Nc2ccc(Cl)c(F)c2)c2ccc(C[NH3+])cc2N1C(=O)OCC(O)CO ; NC(=[NH2+])NC[C@@H]1[C@@H](NC(=O)C(=O)Nc2ccc(Cl)c(F)c2)c2ccc(C[NH3+])cc2N1C(=O)OCc1ccc(Br)cc1] 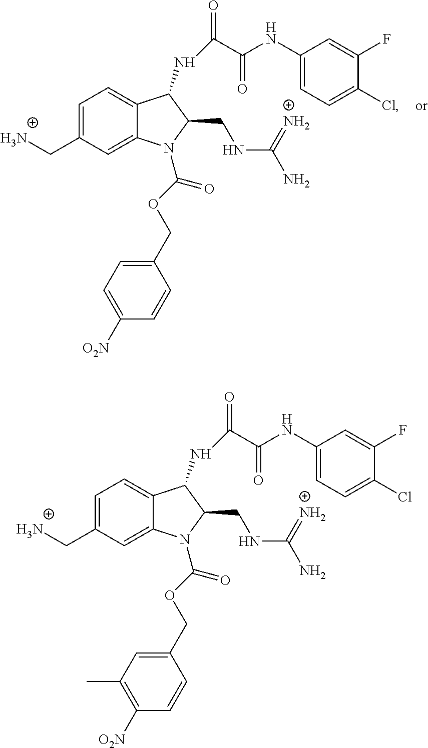 [SMILES: NC(=[NH2+])NC[C@@H]1[C@@H](NC(=O)C(=O)Nc2ccc(Cl)c(F)c2)c2ccc(C[NH3+])cc2N1C(=O)OCc1ccc([N+](=O)[O-])cc1 ; Cc1cc(COC(=O)N2c3cc(C[NH3+])ccc3[C@H](NC(=O)C(=O)Nc3ccc(Cl)c(F)c3)[C@H]2CNC(N)=[NH2+])ccc1[N+](=O)[O-]]
or a pharmaceutically acceptable salt thereof.
